(19) United States
(12) Patent Application Publication
(10) Pub. No.: US 2025/0082212 A1
(43) Pub. Date: Mar. 13, 2025

(54) SYSTEM AND METHOD FOR BLOOD PRESSURE ESTIMATE BASED ON PTT FROM THE FACE

(71) Applicant: BINAH.AI LTD, Tel Aviv (IL)

(72) Inventors: David MAMAN, Tel Aviv (IL); Konstantin GEDALIN, Tel Aviv (IL); Michael MARKZON, Tel Aviv (IL)

(21) Appl. No.: 18/580,598

(22) PCT Filed: Jul. 18, 2022
(86) PCT No.: PCT/IL2022/050771

§ 371 Date: Jan. 18, 2024

Related U.S. Application Data
(60) Provisional application No. 63/223,298, filed on Jul. 19, 2021.

(51) Int. Cl. A61B 5/021; A61B 5/00; A61B 5/0205; A61B 5/024; A61B 5/091; A61B 5/1455; A61B 5/16; G06T 7/00; G06T 7/40; G06T 7/62; G16H 10/60; G16H 30/40

(52) U.S. Cl. CPC A61B 5/02125; A61B 5/0064; A61B 5/02416; A61B 5/6898; A61B 5/7203; A61B 5/7278; G06T 7/0012; G06T 7/40; G06T 7/62; G16H 10/60; G16H 30/40; A61B 5/0205; A61B 5/091; A61B 5/14552; A61B 5/165; A61B 2560/0443; G06T 2207/10016; G06T 2207/20076; G06T 2207/20084; G06T 2207/30088; G06T 2207/30201

(57) ABSTRACT

A new system and method is provided for blood pressure measurements that use an accuracy-improved pulse rate detection, which includes determining PTT (pulse transit time). Various aspects contribute to the greater accuracy, including but not limited to pre-processing of the camera output/input, extracting the pulsatile signal from the preprocessed camera signals, followed by post-filtering of the pulsatile signal. This improved information is then used for accurate BP determination, which is not possible with inaccurate methods for optical pulse rate detection.

### FIELD OF THE INVENTION

The present invention is of a system and method for blood pressure (BP) measurements as determined from optical data, and in particular, for such a system and method for determining such measurements from video data of a subject with a plurality of cameras.

### BACKGROUND OF THE INVENTION

Heart rate measurement devices date back to 1870's with the first electrocardiogram (ECG or EKG), measuring the electric voltage changes due to heart cardiac cycle (or heart beat). The EKG signal is com-posed from three main components: P wave which represents the atria depolarization; the QRS complex represents ventricles depolarization; and T wave represents ventricles re-polarization.

A second pulse rate detection technique is optical measurement that detects blood volume changes in the microvascular bed of tissue named photo-plethysmography (PPG 09o1). In PPG measurement the peripheral pulse wave characteristically exhibits systolic and diastolic peaks. The systolic peak is a result of direct pressure wave traveling from the left ventricle to the periphery of the body, and the diastolic peak (or inflection) is a result of reflections of the pressure wave by arteries of the lower body.

There are two categories of PPG based devices: contact-based and remote (rPPG). The contact-based device typically is used on the finger and measures the light reflection typically at red and IR (infrared) wave-lengths. On the other hand, the remote PPG device measures the light reflected from the skin surface, typically of the face. Most rPPG algorithms use RGB cameras, and do not use IR cameras.

The PPG signal comes from the light-biological tissue interaction, thus depends on (multiple) scattering, absorption, reflection, transmission and fluorescence. Different effects are important depending on the type of device, for contact based or remote PPG measurement. In rPPG analysis a convenient first order decomposition of the signal is to intensity fluctuations, scattering (which did not interact with biological tissues), and the pulsatile signal. The instantaneous pulse time is set from the R-time in EKG measurement or the systolic peak in a PPG measurement. The EKG notation is used to refer to the systolic peak of the rPPG measurement as R time. The instantaneous heart rate is evaluated from the difference between successive R times, RR(n)=R(n)−R(n−1), as 60/RR(n) in beats per minute.

An additional measurement that can be important for health is pulse transit time (PTT). PTT is the time it takes the Pulse Pressure (PP) waveform to propagate through a length of the arterial tree. The velocity of the pressure wave is referred to as the pulse wave velocity (PWV); it can be estimated by determining the PTT. The pulse pressure waveform results from the ejection of blood from the left ventricle and moves with a velocity much greater than the forward movement of the blood itself. It currently requires a combination of an ECG (electrocardiogram) and a finger-mounted device to record the pulse waveform at the fingertip. However these devices are difficult to use and require supervision by medical personnel.

### BRIEF SUMMARY OF THE INVENTION

Accurate optical pulse rate detection unfortunately has suffered from various technical problems. The major difficulty is the low signal to noise achieved and therefore failure to detect the pulse rate. Accurate pulse rate detection is needed to create such additional measurements as PTT, which requires an accurate measurement of pulse signal initiation and also pulse waveforms at a plurality of different tissue locations, such as for example at two points on the face, and/or at a point on the face and a fingertip of the subject.

As described in greater detail below, obtaining HR signal measurements from signals at two different tissue locations on the body may be used for detecting the initiation of the pulse. The combination of detection of the pulse waveform initiation and fingertip pulse waveform measurements support determination of PTT. However, blood pressure measurements may be determined only from analysis of optical data from the face of the subject, if optical data is taken from two different points on the face of the subject, such as for example from the chin and the forehead of the user.

The presently claimed invention, in at least some embodiments, provides a new system and method for blood pressure measurements that uses an accuracy-improved pulse rate detection. Various aspects contribute to the greater accuracy, including but not limited to pre-processing of the camera output/input, extracting the pulsatile signal from the preprocessed camera signals, followed by post-filtering of the pulsatile signal. This improved information may then be used for such analysis as heart rate variability (HRV) determination, which is not possible with inaccurate methods for optical pulse rate detection.

As noted previously, currently the determination of the PTT requires a combination of an ECG (electrocardiogram) and a finger-mounted device to record the pulse waveform at the fingertip. The ECG is used to determine the proximal timing reference for PTT measurements, by using activity of the heart as measured by the R-wave. Esmaili et al (“Non-invasive Blood Pressure Estimation Using Phonocardiogram”, 2017 IEEE International Symposium on Circuits and Systems (DOI: 10.1109/ISCAS.2017.8050240)) proposed using phonocardiogram (PCG) instead of ECG for the proximal timing reference for PTT measurements. PCG is produced due to the opening and closing of heart valves, which each create a sound. The S1 peak of PCG is a sound which can be detected, formed as blood leaves the heart. The authors proposed that the S1 peak could be used in place of the ECG R-peak for measuring PTT. As an actual sound, it can be collected with a microphone.

However, this arrangement still has many drawbacks. For example, the finger-mounted device is specialty hardware which is not always readily available. The ECG is even more complex as a device. Using a microphone would potentially reduce the complexity of the heart timing measurement, but introduces other problems, such as issues of background noise and even other noises from the body.

These disadvantages may be overcome with the following exemplary implementation. In order to achieve this goal, a data acquisition hardware with suitable sampling rate resolution is used. Such hardware is preferably able to continuously measure heart rate (HR). Moreover, the hardware clock is preferably synchronized to produce minimum and constant sampling delay and jitter. All acquired signals then represent a time series, which may be used directly or indirectly HR measurement. This setup is robust in the sense that the relative time delay for detection of HR remains constant according to the differential locations in which the sensors that are used to obtain the signals are placed. In this situation, propagation of the same pulse will still provide the relative time delay. The distances between pairs of sensors are preferably chosen to produce a reliable time delay, such that more preferably the delay in terms of HR pulse detection is larger than the hardware delay.

As noted herein, a non-limiting example of such a pair of sensors relates to two different sets of optical data, taken from two different tissue locations on the body. By “two” it is meant a plurality of locations and sets of optical data, although optionally reference is made to a pair in terms of a preferred example of a minimum set of different data locations. Such different sets of optical data may for example be taken with a plurality of cameras.

A non-limiting example of such an implementation would be a first video camera to obtain optical data from a first point on a face of the user (or subject), such as for example the forehead, and a second video camera to obtain optical data from another point, such as the chin. As a non-limiting example, a device having two different cameras could be used, including but not limited to a smart phone, cellular telephone, tablet, mobile phone, or other computational device having two cameras. Optionally one camera is used rather than two cameras.

Optionally, a calibration procedure is performed to more accurately determine the body tissue locations being measured. For example, the calibration procedure may include a supervised physical exercise. In addition, the above measurement of PTT may be used for more accurate determination of blood pressure (BP), including without limitation better determination of BP variability.

A non-limiting example of such an implementation would be a single video camera (preferably) or a plurality of cameras to obtain optical data from multiple regions of a face (i.e., right forehead and left cheek or vice versa) of the user (or subject). Optionally, optical data may be obtained from the face as a whole, and then optionally also from one additional region of the face, or a left or right portion of that region. For example and without limitation, the additional region may comprise the forehead, or a left, center or right portion thereof; a left or right cheek; a chin portion; and so forth. The optical data may be obtained simultaneously over a duration of time from the multiple regions or may be obtained at staggered time intervals from each region. As a non-limiting example, a device having two different cameras could be used, including but not limited to a smart phone, cellular telephone, tablet, mobile phone, or other computational device having two cameras.

The user could hold the device with the camera, such that a camera is able to obtain optical data of the user's face. Such a camera may comprise a front camera of a mobile device, such as a smart phone for example. In this example, optical signals are obtained from the front camera from different regions of the user's face over a duration of time or staggered time intervals, producing a time-series of PPG signals for each region. A delay between time-series of the PPG signals of two regions is calculated according to a predetermined time interval, for example 30 seconds. These time-series of the PPG, along with the delay, can be used to derive PTT values through the natural delay between the pulse signals detected at the two different regions of the face.

After obtaining the above measurement of PTT, the PTT measurement may be used for more accurate determination of blood pressure (BP), including without limitation better determination of BP variability.

Optionally in combination with any method or a portion thereof as described herein, said detecting said optical data from said skin of the face comprises determining a plurality of face or fingertip boundaries, selecting the face or fingertip boundary with the highest probability and applying a histogram analysis to video data from the face or fingertip. Optionally said determining said plurality of face or fingertip boundaries comprises applying a multi-parameter convolutional neural net (CNN) to said video data to determine said face or fingertip boundaries. Optionally the method may further comprise combining analyzed data from images of the face and fingertip to determine the physiological measurement.

Implementation of the method and system of the present invention involves performing or completing certain selected tasks or steps manually, automatically, or a combination thereof. Moreover, according to actual instrumentation and equipment of preferred embodiments of the method and system of the present invention, several selected steps could be implemented by hardware or by software on any operating system of any firmware or a combination thereof. For example, as hardware, selected steps of the invention could be implemented as a chip or a circuit. As software, selected steps of the invention could be implemented as a plurality of software instructions being executed by a computer using any suitable operating system. In any case, selected steps of the method and system of the invention could be described as being performed by a data processor, such as a computing platform for executing a plurality of instructions.

Although the present invention is described with regard to a “computing device”, a “computer”, or “mobile device”, it should be noted that optionally any device featuring a data processor and the ability to execute one or more instructions may be described as a computer, including but not limited to any type of personal computer (PC), a server, a distributed server, a virtual server, a cloud computing platform, a cellular telephone, an IP telephone, a smartphone, or a PDA (personal digital assistant). Any two or more of such devices in communication with each other may optionally comprise a “network” or a “computer network”.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A and 1B show exemplary non-limiting illustrative systems for obtaining video data of a user and for analyzing the video data to determine PTT and to determine BP;

FIGS. 2A and 2B show non-limiting exemplary methods for performing signal analysis for PTT;

FIGS. 3A and 3B show non-limiting exemplary methods for enabling the user to use the app to obtain biological statistics;

FIG. 4 shows a non-limiting exemplary process for creating detailed biological statistics;

FIGS. 5A-5E show a non-limiting, exemplary method for obtaining video data and then performing the initial processing;

FIG. 6A relates to a non-limiting exemplary method for pulse rate estimation and determination of the rPPG, while FIGS. 6B-6C relate to some results of this method;

FIG. 7 relates to a non-limiting, exemplary implementation of a method for PTT determination and for BP determination according to at least some embodiments of the present invention; and

FIG. 8 relates to a non-limiting, exemplary implementation of a method for PTT determination and for BP determination with a plurality of face portions according to at least some embodiments of the present invention.

### DESCRIPTION OF AT LEAST SOME EMBODIMENTS

Pulse transit time (PTT) requires direct or indirect measurement of R waves, for example through rPPG, and also determination of the pulse waveforms at another tissue point, such as another point on the face and/or the fingertip, which may also be measured through some type of PPG. Preferably a combination of rPPG and PPG pulse signal measurements are performed, in which the rPPG is taken from a body tissue location that is different from the PPG signal measurement location. For example the PPG signal may be obtained by placing a fingertip against a video camera. PPG signal measurements are known in the art and may be implemented as described below, or according to other PPG signal measurement devices and systems.

However, rPPG measurements have many inherent challenges. A key underlying problem for rPPG mechanisms is accurate face and finger detection, and/or accurate detection of two different points on the face, and precise skin surface selection suitable for analysis. The presently claimed invention overcomes this problem for face, finger and skin detection based on neural network methodology. Non-limiting examples are provided below. Preferably, for the skin selection, a histogram based algorithm is used. Applying this procedure on part of the video frame containing face only, the mean values for each channel, Red, Green, and Blue (RGB) construct the frame data. When using above procedures continuously for consequent video frames, the time series of RGB data is obtained. Each element of these time series represented by RGB values is obtained frame by frame, with time stamps used to determine elapsing time from the first occurrence of the first element. Then, the rPPG analysis begins when the total elapsed time reaches the averaging period used for the pulse rate estimation defined external parameter, for a complete time window (Lalgo). Taking into account the variable frame acquisition rate, the time series data has to be interpolated with respect to the fixed given frame rate.

After interpolation, a pre-processing mechanism is applied to construct a more suitable three dimensional signal (RGB). Such pre-processing may include for example normalization and filtering. Following pre-processing, the rPPG trace signal is calculated, including estimating the mean pulse rate and the initiation of the pulse waveform. If two different points on the face are used, such as optical data from the forehead and chin for example, two rPPG extractions are preferably performed at the same time.

A similar process may be followed for images of the fingertip, for example for images taken with the rear facing camera of a mobile device, such as a smart phone for example.

In order for PTT to be determined, the delay between the pulse waveform measurements taken at the two different locations needs to be accurately determined. Therefore, the sensors involved in the rPPG and PPG measurements need to be accurately synchronized. Hardware synchronization is preferred, as accurate PTT measurements require accurate synchronization between the timing of obtaining the signals, so that the relative delay between the two pulse measurements can be accurately determined. The determined PTT is used for BP determination.

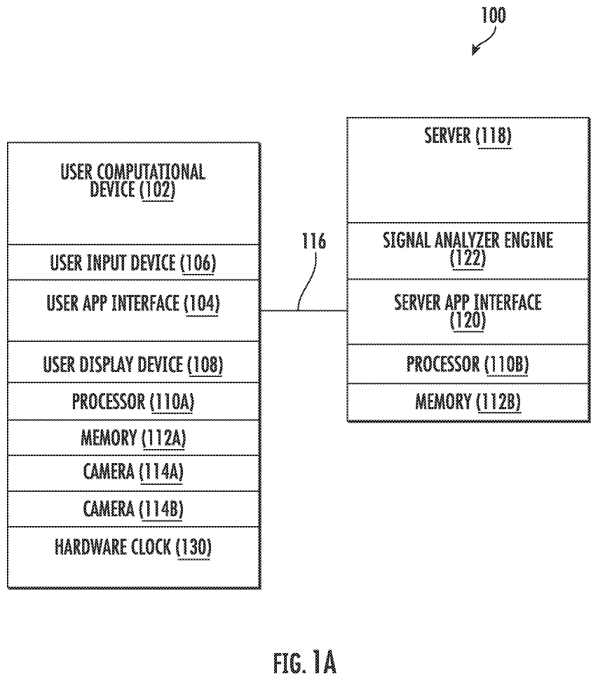

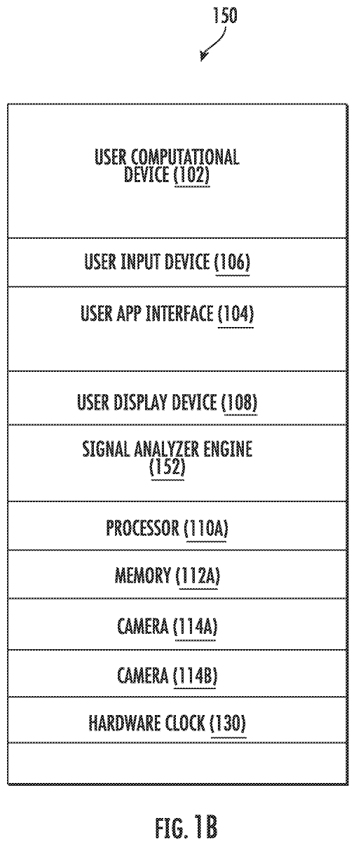

Turning now to the drawings, FIGS. 1A and 1B show exemplary non-limiting illustrative systems for obtaining video data of a user and for analyzing the video data to determine one or more biological signals, for determining PTT and for determining BP.

FIG. 1A shows a system 100 featuring a user computational device 102, communicating with a server 118. The user computational device 102 preferably communicates with a server 118 through a computer network 116. User computational device 102 preferably includes user input device 106, which may include, for example, a pointing device such as a mouse, keyboard, and/or other input device.

In addition, user computational device 102 preferably includes a plurality of cameras 114, shown as camera 114A and camera 114B. For example, camera 114A may be used for obtaining video data of a face of the user. Camera 114B may be used for obtaining video data of a finger tip of the user. For the latter, the finger is preferably pressed against camera 114B. Alternatively, a single camera, such as camera 114A for example, may be used to obtain video data of a plurality of different parts of the face, such as video data of the forehead and of the chin for example.

Each or both of cameras 114A and 114B may also be separate from the user computational device. The user interacts with a user app interface 104, for providing commands for determining the type of signal analysis, for starting the signal analysis, and for also receiving the results of the signal analysis.

For example, the user may, through user computational device 102, start recording video data of the face of the user through camera 114A, either by separately activating camera 114A, or by recording such data by issuing a command through user app interface 104. Similarly, the user may start recording data of the fingertip of the user through camera 114B, either by separately activating camera 114B, or by recording such data by issuing a command through user app interface 104. Alternatively, the user may start recording video data of the face of the user through camera 114A only.

In an embodiment, user computational device 102 comprises a mobile communication device, such as a smart phone for example. For this type of device, typically there is a front camera and a rear camera. User app interface 104 preferably enables both cameras to be activated simultaneously or near-simultaneously. Optionally either of cameras 114A and 114B may be the front or rear camera. For ease of use of user app interface 104, preferably camera 114A, capturing the face of the user, is the front camera (mounted over or in the same orientation as the display screen, shown as user display device 108, while camera 114B, capturing the fingertip of the user, is the rear camera.

Next, the video data is preferably sent to server 118, where it is received by server app interface 120. It is then analyzed by signal analyzer engine 122. Signal analyzer engine 122 preferably includes detection of the face in the video signals from camera 114A, followed by skin detection. As described in detail below, various non-limiting algorithms are preferably applied to support obtaining the pulse signals from this information. In addition, the signals from camera 114B are preferably analyzed according to PPG signal analysis, to detect the pulse waveform and its timing at the fingertip. Signal analyzer engine 122 may also be implemented for fingertip detection in the video data to support such analysis.

Next, the pulse signals are preferably analyzed according to time, frequency and non-linear filters to support the determination of pulse waveform timing. For example, the timing of the two signals is preferably determined according to hardware synchronization. Such synchronization may for example be determined through a hardware clock 130. Further analyses may then be performed to calculate PTT.

User computational device 102 preferably features a processor 110A, and a memory 112A. Server 118 preferably features a processor 1101B, and a memory 112B.

As used herein, a processor such as processor 110A or 110B generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory, such as memory 112A or 112B in this non-limiting example. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

In addition, user computational device 102 may feature user display device 108 for displaying the results of the signal analysis, the results of one or more commands being issued and the like.

In another embodiment using only a front camera, user computational device 102 comprises a mobile communication device, such as a smart phone for example. For this type of device, typically there is a front camera and a rear camera. User app interface 104 preferably enables the front camera to be activated. Optionally either of cameras 114A and 114B may be the front or rear camera. For ease of use of user app interface 104, preferably camera 114A, capturing the face of the user, is the front camera (mounted over or in the same orientation as the display screen, shown as user display device 108, while camera 114B is the rear camera. Again, optionally only the front camera is used to capture video data of the face at a plurality of points.

The front camera may for example capture optical data (i.e., video data) from multiple regions of the user's face (i.e., right forehead and left cheek or vice versa). The video data may be obtained simultaneously over a duration of time from the multiple regions or may be obtained at staggered time intervals from each region.

Next, the video data is preferably sent to server 118, where it is received by server app interface 120. It is then analyzed by signal analyzer engine 122. Signal analyzer engine 122 preferably includes detection of the face in the video signals from camera 114A.

As described in detail below, various non-limiting algorithms are preferably applied to support obtaining the pulse signals from this information. In addition, the signals from camera 114A are preferably analyzed according to PPG signal analysis to detect the pulse waveform and its timing in different facial regions. Signal analyzer engine 122 may also be implemented for facial detection in the video data to support such analysis.

Next, the pulse signals are preferably analyzed according to time, frequency and non-linear filters to support the determination of pulse waveform timing. In addition, a delay between time-series of the PPG signals of two regions is calculated according to a time interval, for example 30 seconds. These time-series of the PPG, along with the delay, can be used to derive PTT values through the natural delay between the pulse signals detected at the plurality of different regions of the face.

After obtaining the above measurement of PTT, the PTT measurement may be used for more accurate determination of blood pressure (BP), including without limitation better determination of BP variability.

FIG. 1B shows a system 150, in which the above described functions are performed by user computational device 102. For either of FIG. 1A or 1B, user computational device 102 may comprise a mobile phone. In FIG. 1B, the previously described signal analyzer engine is now operated by user computational device 102 as signal analyzer engine 152. Signal analyzer engine 152 may have the same or similar functions to those described for signal analyzer engine in FIG. 1A. In FIG. 1B, user computational device 102 may be connected to a computer network such as the internet (not shown) and may also communicate with other computational devices. In at least some embodiments, some of the functions are performed by user computational device 102 while others are performed by a separate computational device, such as a server for example (not shown in FIG. 1B, see FIG. 1A).

Optionally, memory 112A or 112B is configured for storing a defined native instruction set of codes. Processor 110A or 1101B is configured to perform a defined set of basic operations in response to receiving a corresponding basic instruction selected from the defined native instruction set of codes stored in memory 112A or 112B.

Optionally memory 112A or 112B stores a first set of machine codes selected from the native instruction set for analyzing the optical data to select data related to the face of the subject, a second set of machine codes selected from the native instruction set for detecting optical data from a skin of the face, a third set of machine codes selected from the native instruction set for determining a time series from the optical data by collecting the optical data until an elapsed period of time has been reached and then calculating the time series from the collected optical data for the elapsed period of time; and a fourth set of machine codes selected from the native instruction set for calculating the physiological signal from the time series.

Optionally memory 112A or 112B further comprises a fifth set of machine codes selected from the native instruction set for detecting said optical data from said skin of the face comprises determining a plurality of face boundaries, a sixth set of machine codes selected from the native instruction set for selecting the face boundary with the highest probability and a seventh set of machine codes selected from the native instruction set for applying a histogram analysis to video data from the face.

Optionally memory 112A or 112B further comprises an eighth set of machine codes selected from the native instruction set for applying a multi-parameter convolutional neural net (CNN) to said video data to determine said face boundaries.

Optionally memory 112A or 112B stores a ninth set of machine codes selected from the native instruction set for analyzing the optical data to select data related to the fingertip of the subject, a tenth set of machine codes selected from the native instruction set for detecting optical data from a skin of the fingertip, a eleventh set of machine codes selected from the native instruction set for determining a time series from the optical data by collecting the optical data until an elapsed period of time has been reached and then calculating the time series from the collected optical data for the elapsed period of time; and a twelfth set of machine codes selected from the native instruction set for calculating the physiological signal from the time series.

Optionally memory 112A or 112B further comprises a thirteenth set of machine codes selected from the native instruction set for detecting said optical data from said skin of the fingertip comprises determining a plurality of fingertip boundaries, a fourteenth set of machine codes selected from the native instruction set for selecting the fingertip boundary with the highest probability and a fifteenth set of machine codes selected from the native instruction set for applying a histogram analysis to video data from the fingertip.

Optionally memory 112A or 112B further comprises a sixteenth set of machine codes selected from the native instruction set for applying a multi-parameter convolutional neural net (CNN) to said video data to determine said fingertip boundaries. Additionally or alternatively, the fingertip is pressed against the camera so only skin detection is performed, rather than fingertip detection.

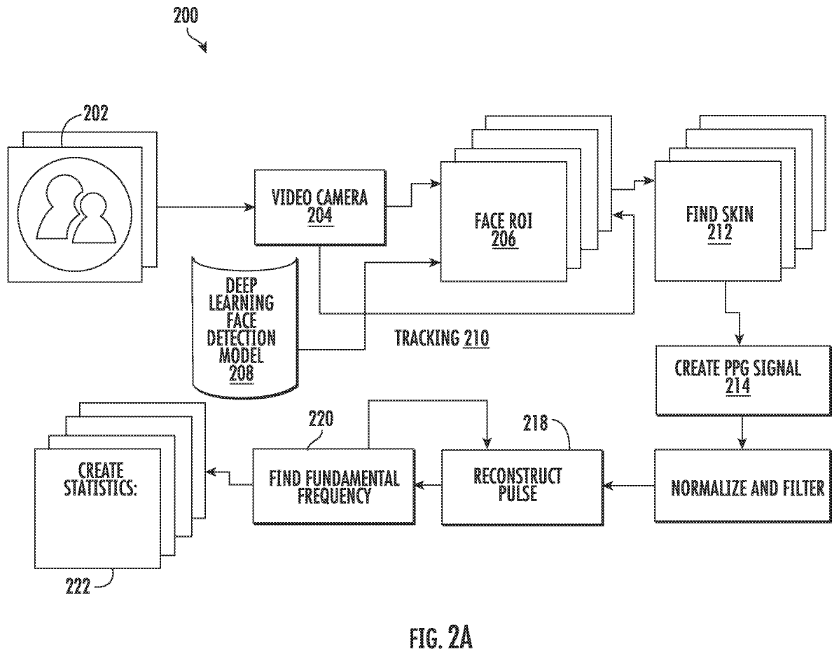

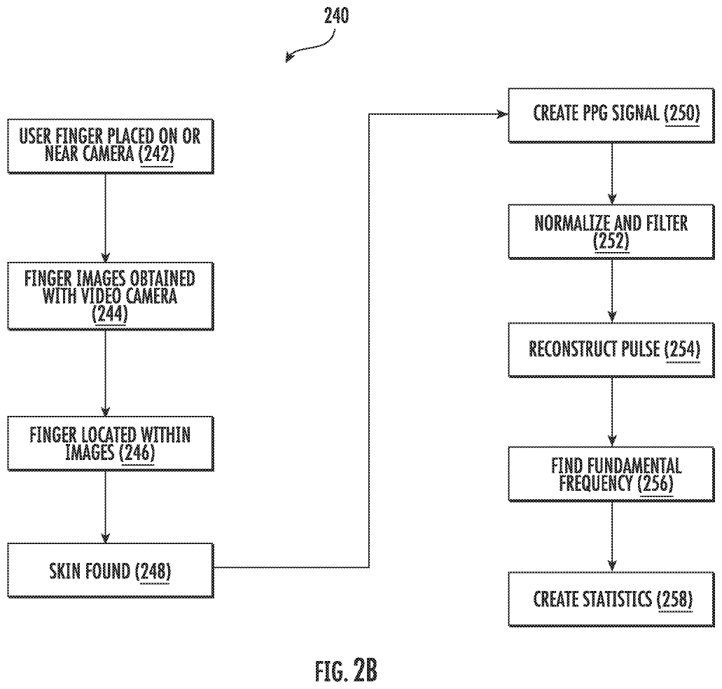

FIG. 2A shows a non-limiting exemplary method for performing signal analysis, for detecting the pulse signal and other relevant signals from the face of the user. A process 200 begins by initiating the process of obtaining data at block 202, for example, by activating a video camera 204. Face recognition is then optionally performed at 206, to first of all locate the face of the user. This may, for example, be performed through a deep learning face detection module 208, and also through a tracking process 210. It is important to locate the face of the user, as the video data is preferably of the face of the user in order to obtain the most accurate results for signal analysis. Tracking process 210 is based on a continuous features matching mechanism. The features represent a previously detected face in a new frame. The features are determined according to the position in the frame and from the output of an image recognition process, such as a CNN (convolutional neural network). When only one face appears in the frame, tracking process 210 can be simplified to face recognition within the frame.

As a non-limiting example, optionally, a Multi-task Convolutional Network algorithm is applied for face detection which achieves state-of-the-art accuracy under real-time conditions. It is based on the network cascade that was introduced in a publication by Li et al (Haoxiang Li, Zhe Lin, Xiaohui Shen, Jonathan Brandt, and Gang Hua. A convolutional neural network cascade for face detection. In The IEEE Conference on Computer Vision and Pattern Recognition (CVPR), June 2015).

Next, the skin of the face of the user is located within the video data at 212. The skin of the user is preferably located at a plurality of different portions, such as for example the forehead and skin, which are analyzed separately. Preferably, for the skin selection, a histogram based algorithm is used. Applying this procedure on part of the video frame containing the face only, as determined according to the previously described face detection algorithm, the mean values for each channel, Red, Green, and Blue (RGB) are preferably used to construct the frame data. When using above procedures continuously for consequent video frames, a time series of RGB data is obtained. Each frame, with its RGB values, represents an element of these time series. Each element has a time stamp determined according to elapsed time from the first occurrence. The collected elements may be described as being in a scaled buffer having L algo elements. The frames are preferably collected until sufficient elements are collected. The sufficiency of the number of elements is preferably determined according to the total elapsed time. The rPPG analysis of 214 begins when the total elapsed time reaches the length of time required for the averaging period used for the pulse rate estimation. The collected data elements may be interpolated. Following interpolation, the pre-processing mechanism is preferably applied to construct a more suitable three dimensional signal (RGB).

A plurality of PPG signals is created at 214 from the three dimensional signal and specifically from the elements of the RGB data, at the plurality of portions of the face. For example, the pulse rate may be determined from a single calculation or from a plurality of cross-correlated calculations, as described in greater detail below. This may be then normalized and filtered at 216, and may be used to reconstruct ECG at 218. A fundamental frequency is found at 220, and the statistics are created such as heart rate, pulse signal timing and so forth at 222.

FIG. 2B shows a similar, non-limiting, exemplary method for analyzing video data of the fingertip of the user, for example from the rear camera of a mobile device as previously described. Again, preferably this video data is captured simultaneously or near simultaneously with the video data of the face. In a method 240, the method begins by placing the fingertip of the user on or near the camera at 242. If near the camera, then the fingertip needs to be visible to the camera. This placement may be accomplished for example in a mobile device, by having the user place the fingertip on the rear camera of the mobile device, while the front camera is used to take images of the face of the user, “selfie” style. The cameras are already in a known geometric position, which encourages correct placement of the fingertip and face.

At 244, images of the finger, and preferably of the fingertip, are obtained with the camera. Next the finger, and preferably the fingertip, is located within the images at 246. This process may be performed as previously described with regard to location of the face within the images. However, if a neural net is used, it will need to be trained specifically to locate fingers and preferably fingertips. Hand tracking from optical data is known in the art; a modified hand tracking algorithm could be used to track fingertips within a series of images.

At 248, the skin is found within the finger, and preferably fingertip, portion of the image. Again, this process may be performed generally as described above for skin location, optionally with adjustments for finger or fingertip skin. Again, preferably a histogram based method is used, and images are collected until enough are available to perform PPG/rPPG at 250. Once this data has been obtained, steps 250-256 may be performed as described above with regard to steps 214-220. However step 258 also includes determination of the pulse waveform and also of the relative time difference between the pulse signal timing at the two different locations, for example according to a synchronized hardware clock.

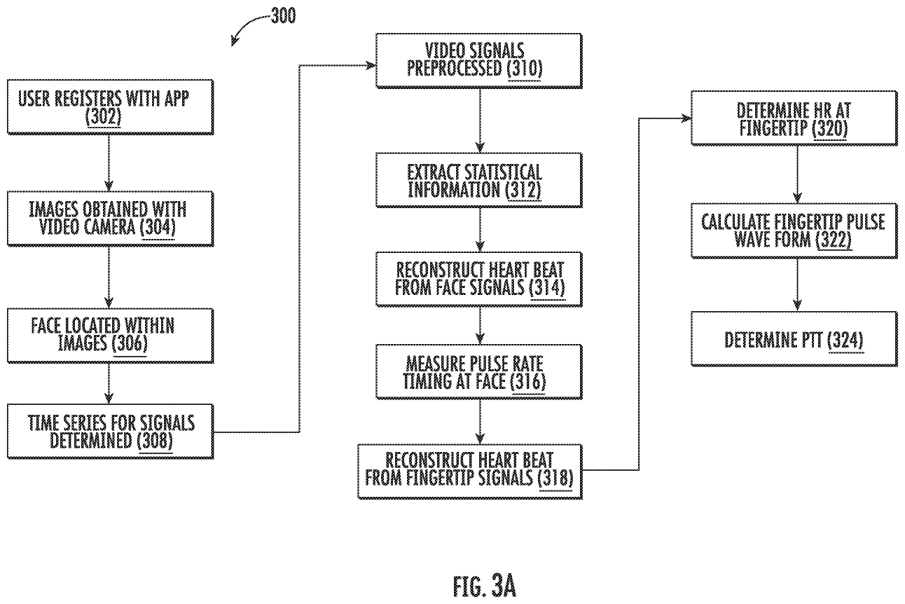

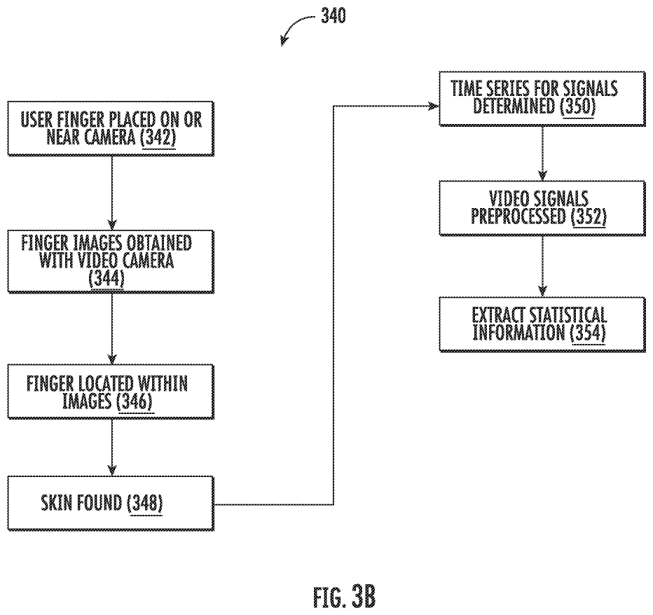

FIGS. 3A and 3B show non-limiting exemplary methods for enabling the user to use the app to obtain biological statistics. In a method 300, the user registers with the app at 302. Next, images are obtained with the video camera, for example as attached to or formed with user computational device at 304. The video camera is preferably a RGB camera as described herein.

The face is located within the images 306. This may be performed on the user computational device, at a server, or optionally at both. Furthermore, this process may be performed as previously described, with regard to a multi-task convolutional neural net. Skin detection is then performed, by applying a histogram to the RGB signal data. Only the video data relating to light reflected from the skin is preferably analyzed for optical pulse detection and HRV determination.

The time series for the signals are determined at 308, for example as previously described. Taking into account the variable frame acquisition rate, the time series data is preferably interpolated with respect to the fixed given frame rate. Before running the interpolation procedure, preferably the following conditions are analyzed so that interpolation can be performed. First, preferably the number of frames is analyzed to verify that after interpolation and pre-processing, there will be enough frames for the rPPG analysis.

Next, the frames per second are considered, to verify that the measured frames per second in the window is above a minimum threshold. After that, the time gap between frames, if any, is analyzed to ensure that it is less than some externally set threshold, which for example may be 0.5 seconds.

If any of the above conditions are not satisfied, then the procedure preferably terminates with full data reset and restarts from the last valid frame, for example to return to 304 as described above.

Next the video signals are preferably pre-processed at 310, following interpolation. The pre-processing mechanism is applied to construct a more suitable three dimensional signal (RGB). The pre-processing preferably includes normalizing each channel to the total power; scaling the channel value by its mean value (estimated by low pass filter) and subtracting by one; and then passing the data through a Butterworth band pass IIR filter.

Statistical information is extracted at 312, including the timing of the signals in relation to a hardware clock. A heartbeat is then reconstructed at 314 from the face optical signals, relating to the entire face or alternatively a portion of the face. The pulse rate timing is then determined from the face signals at 316.

Now the heart beat is reconstructed from the optical signals from a second portion of the face and/or from the fingertip as a second tissue location, including with regard to the timing of the signals in relation to a hardware clock, at 318. The HR timing at the second tissue location is then determined at 320. The wave pulse form of the second tissue location is then calculated at 322, including with regard to a differential timing, or delay, in relation to the pulse rate timing as determined from the first tissue location signals. Determination of the differential timing is preferably assisted by the synchronization of the signals through a hardware clock. The PTT is then determined according to the differential timing, at 324.

The heartbeat may be reconstructed from the video data capture from a plurality of regions of the user's face, such as from the forehead and chin of the face. Similarly, the HR timing is determined at 320 by using the calculated delay between the time-series of the PPG signal and the video date obtained from the two different facial regions. The wave pulse form is then calculated at 322 in relation to the pulse rate timing as determined from the face signals from both facial regions. The PTT is then determined according to the differential timing and pulse waveform detection from the video data. After obtaining the above measurement of PTT, the PTT measurement may be used for more accurate determination of blood pressure (BP), including without limitation better determination of BP variability.

FIG. 3B shows an exemplary, non-limiting method for obtaining and analyzing the fingertip optical signals which may then be fed to the above process at 314, if the fingertip comprises the second tissue location for providing signals. FIG. 3B shows a similar, non-limiting, exemplary method for analyzing video data of the fingertip of the user, for example from the rear camera of a mobile device as previously described. This process may be used for example if sufficient video data cannot be captured from the front facing camera, for the face of the user. Optionally both methods may be combined.

In a method 340, the method begins by placing the fingertip of the user on or near the camera at 342. If near the camera, then the fingertip needs to be visible to the camera. This placement may be accomplished for example in a mobile device, by having the user place the fingertip on the rear camera of the mobile device. The camera is already in a known geometric position in relation to placement of the fingertip, which encourages correct placement of the fingertip in terms of collecting accurate video data. Optionally the flash of the mobile device may be enabled in a longer mode (“torch” or “flashlight” mode) to provide sufficient light. Enabling the flash may be performed automatically if sufficient light is not detected by the camera for accurate video data of the fingertip to be obtained.

At 344, images of the finger, and preferably of the fingertip, are obtained with the camera. Next the finger, and preferably the fingertip, is located within the images at 346. This process may be performed as previously described with regard to location of the face within the images. However, if a neural net is used, it will need to be trained specifically to locate fingers and preferably fingertips. Hand tracking from optical data is known in the art; a modified hand tracking algorithm could be used to track fingertips within a series of images.

At 348, the skin is found within the finger, and preferably fingertip, portion of the image. Again, this process may be performed generally as described above for skin location, optionally with adjustments for finger or fingertip skin. The time series for the signals are determined at 350, for example as previously described but preferably adjusted for any characteristics of using the rear camera and/or the direct contact of the fingertip skin on the camera. Taking into account the variable frame acquisition rate, the time series data is preferably interpolated with respect to the fixed given frame rate. Before running the interpolation procedure, preferably the following conditions are analyzed so that interpolation can be performed. First, preferably the number of frames is analyzed to verify that after interpolation and pre-processing, there will be enough frames for the rPPG analysis.

Next, the frames per second are considered, to verify that the measured frames per second in the window is above a minimum threshold. After that, the time gap between frames, if any, is analyzed to ensure that it is less than some externally set threshold, which for example may be 0.5 seconds.

If any of the above conditions is not satisfied, then the procedure preferably terminates with full data reset and restarts from the last valid frame, for example to return to 344 as described above.

Next the video signals are preferably pre-processed at 352, following interpolation. The pre-processing mechanism is applied to construct a more suitable three dimensional signal (RGB). The pre-processing preferably includes normalizing each channel to the total power; scaling the channel value by its mean value (estimated by low pass filter) and subtracting by one; and then passing the data through a Butterworth band pass IIR filter. Again, this process is preferably adjusted for the fingertip data. At 354, statistical information is extracted, after which the process may proceed for example as described with regard to FIG. 3A above, from 314.

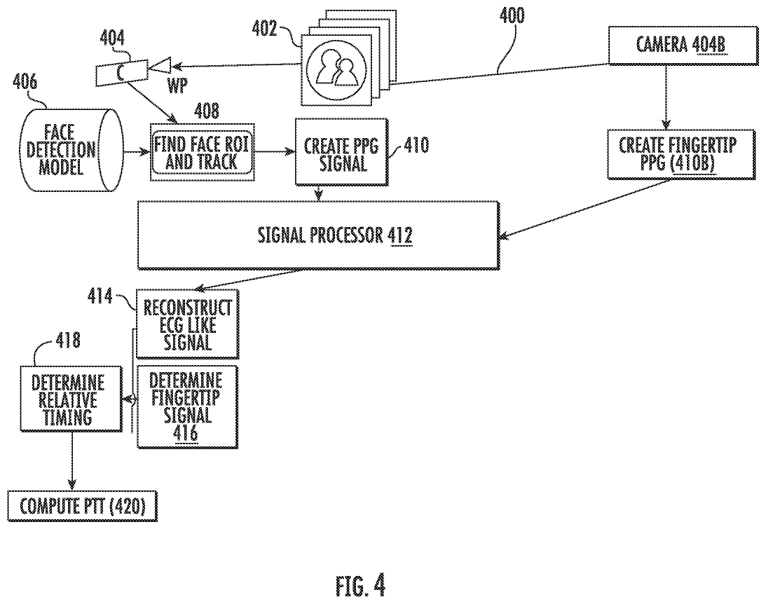

FIG. 4 shows a non-limiting exemplary process for creating detailed biological statistics, including in this non-limiting example, the pulse waveform timing from optical signals taken from a face of the user. In a process 400, user video data is obtained through a user computational device 402, with a camera 404. A face detection model 406 is then used to find the face. For example, after face video data has been detected for a plurality of different face boundaries, all but the highest-scoring face boundary is preferably discarded. Its bounding box is cropped out of the input image, such that data related to the user's face is preferably separated from other video data. Skin pixels are preferably collected using a histogram based classifier with a soft thresholding mechanism, as previously described. From the remaining pixels, the mean value is computed per channel, and then passed on to the rPPG algorithm at 410. This process enables skin color to be determined, such that the effect of the pulse on the optical data can be separated from the effect of the underlying skin color. The process tracks the face at 408 according to the highest scoring face bounding box. A similar process may be used to detect portions of the face of the user, including without limitation the forehead and the chin.

As noted above, this process may be adapted to detect the finger or portion thereof, such as the fingertip for example. Preferably a boundary detecting algorithm is also used to detect the boundaries of the finger or portion thereof, such as the fingertip. The subsequent processes, such as cropping out the bounding box to separate the relevant portion of the user's anatomy, such as the finger or portion thereof, such as the fingertip for example. An adapted histogram based classifier may also be used, given that the relevant portions of the anatomy being detected, such as the fingertip for example, comprise skin. The process at 408 may be adapted if the user presses a fingertip against the rear camera, for example to accommodate a reduced need for tracking, given the direct placement of the fingertip against the rear camera.

Preferably, in a parallel path, a separate camera 404B obtains fingertip optical data, such as video data for example, from a fingertip of the user. Separate camera 404B may be part of user computational device 402.

Next, the PPG signals are created from the face signal data at 410. Following pre-processing, the rPPG trace signal is calculated using Lalgo elements of the scaled buffer. The procedure is described as follows: The mean pulse rate is estimated using a match filter between two rPPG different analytic signals constructed from raw interpolated data (CHROM like and Projection Matrix (PM)). Then the cross-correlation is calculated, on which the mean instantaneous pulse rate may be searched. Frequency estimation is based on non-linear least square (NLS) spectral decomposition with an additional lock-in mechanism. The rPPG signal is then derived from the PM method, applying adaptive Wiener filtering and with initial guess signal to be the dependent on instantaneous pulse rate frequency (vpr): sin(27πvprn). Further, an additional filter in the frequency domain is used to force signal reconstruction. Lastly, the exponential filter applied on instantaneous RR values obtained by procedure discussed in greater detail below.

PPG signals are also preferably obtained from the previously described fingertip video data at 410B.

The signal processor at 412 then preferably performs a number of different functions, based on the PPG signals. These preferably include reconstructing an ECG-like signal at 414, and computing the fingertip pulse signal at 416. In both cases, preferably the timing is measured according to a hardware clock for synchronization as previously described (not shown). At 418, signal processor 412 then preferably determines the relative delay or differential timing between the two sets of pulse signals. Such a differential timing, in combination with the pulse waveform as determined at the fingertip, are preferably used to calculate the PTT at 420.

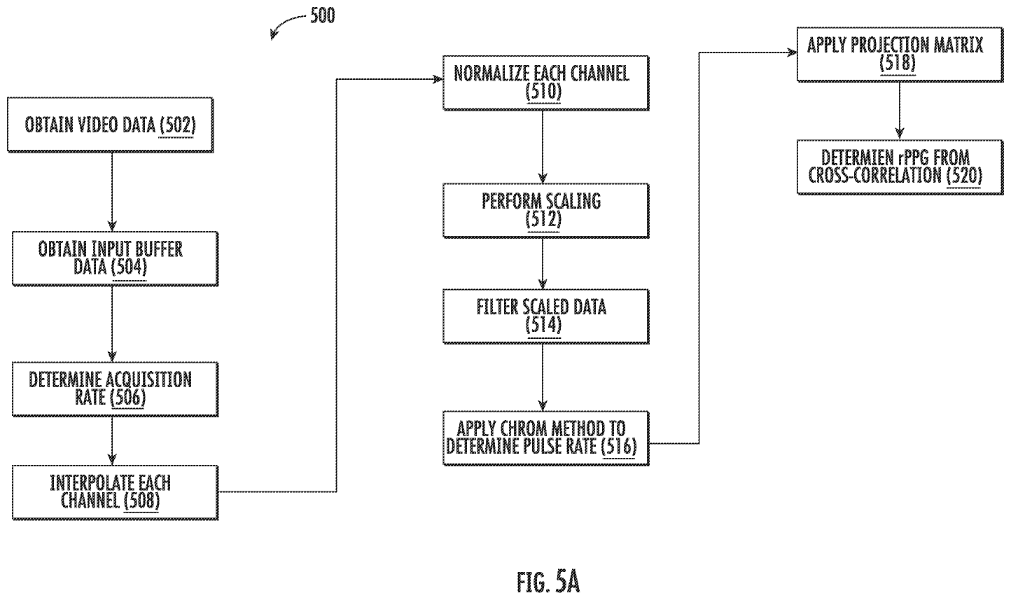

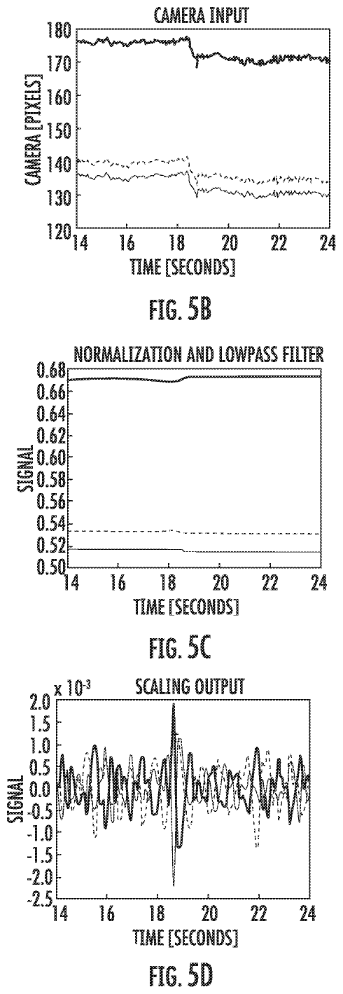

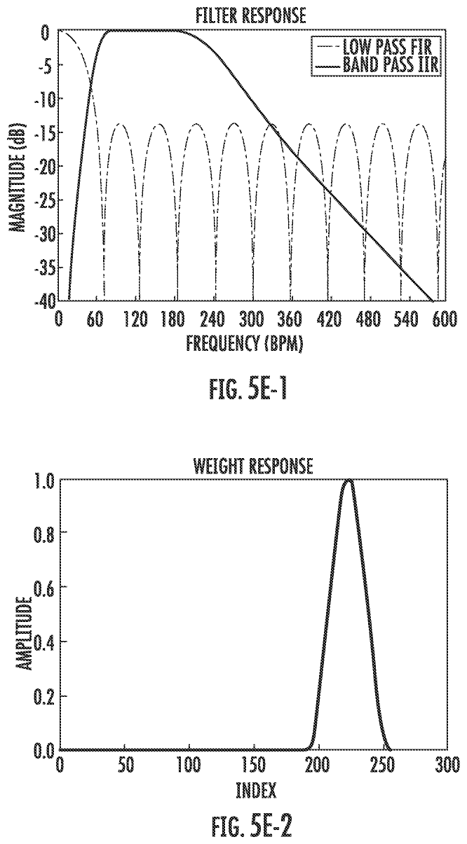

FIGS. 5A-5E show a non-limiting, exemplary method for obtaining video data and then performing the initial processing for determining the rPPG signals from the face optical data, optionally from a plurality of portions of the face, which preferably includes interpolation, pre-processing and rPPG signal determination, with some results from such initial processing. Turning now to FIG. 5A, in a process 500, video data is obtained in 502, for example as previously described.

Next the camera channels input buffer data is obtained at 504, for example as previously described. Next a constant and predefined acquisition rate is preferably determined at 506. For example, the constant and predefined acquisition rate may be set at Δt=1/fps˜=33 ms. At 508, each channel is preferably interpolated separately to the time buffer with the constant and predefined acquisition rate. This step removes the input time jitter. Even though the interpolation procedure adds aliasing (and/or frequency folding), aliasing (and/or frequency folding) has already occurred once the images were taken by the camera. The importance of interpolating into a constant sample rate is that it satisfies a basic assumption of quasi-stationarity of the heart rate in accordance to the acquisition time. The method used for interpolation may for example be based on cubic Hermite interpolation.

FIGS. 5B-5D show data relating to different stages of the scaling procedure. The color coding corresponds to the colors of each channel, i.e. red corresponds to the red channel and so forth. FIG. 5B shows the camera channel data after interpolation.

Turning back to FIG. 5A, at 510-514, after interpolating each of the colored channels (vec(c)), pre-processing is performed to enhance the pulsatile modulations. The pre-processing preferably incorporates three steps. At 510, normalization of each channel to the total power is performed, which reduces noise due to overall external light modulation.

The power normalization is given by

$$C {\to }_{p} = \frac{C \to }{\sqrt{{c}_{r}^{2} + {c}_{g}^{2} + {c}_{b}^{2}}} \qquad ( 1 )$$

with -→c p is the power normalized camera channel vector, and -→c is the interpolated input vector as described. For the sake of brevity, the frame index was removed from both sides.

Next, at 512, scaling is performed. For example, such scaling may be performed by the mean value i and subtracted by one, which reduces effects of stationary light source and its brightness level. The mean value is set by the segment length (Lalgo), but this type of a solution can enhance low frequency components. Alternatively, instead of scaling by the mean value, it is possible to scale by a low pass FIR filter.

Using a low pass filter adds an inherent latency, which requires compensation on M/2 frames. The scaled signal is given by:

$${c}_{s} ( n - \frac{M}{2} ) = \frac{{c}_{p} ( n - \frac{M}{2} )}{{\sum }_{m = 0}^{m = M} b ( m ) {c}_{p} ( n - m )} - 1 \qquad ( 2 )$$

with cs(n) is a single channel scaled value of frame n, and b is the lowpass FIR coefficients. The channel color notation was removed from the above formula for brevity.

At 514, the scaled data is passed through a Butterworth band pass IIR filter.

This filter is defined as:

$$s ( n ) = {\sum }_{m = 0}^{m = {M}_{ff}} b ( m ) {c}_{s} ( n - m ) - {\sum }_{l = 1}^{l = {M}_{fb}} a ( l ) s ( n - 1 ) \qquad ( 3 )$$

The output of the scaling procedure is -→s each new frame adds a new frame with latency for each camera channel. Note that for brevity the frame index n is used but it actually refers to frame n−M/2 (due to the low pass filter).

FIG. 5C shows power normalization of the camera input, plot of the low-pass scaled data before the band-pass filter. FIG. 5D shows a plot of the power scaled data before the band-pass filter. FIG. 5E shows a comparison of the mean absolute deviation for all subjects using the two normalization procedures, with the filter response given as FIG. 5E-1 and the weight response (averaging by the mean) given as FIG. 5E-2. FIG. 5E-1 shows the magnitude and frequency response of the pre-processing filters. The blue line represents the M=33 tap low pass FIR filter, while the red line shows the third order IIR Butterworth filter. FIG. 5E-2 shows the 64 long Hann window weight response used for averaging the rPPG trace.

At 516 the CHROM algorithm is applied to determine the pulse rate. This algorithm is applied by projecting the signals onto two planes defined by

$${S}_{c , 1} = 3 {s}_{r} - 2 {s}_{g} \qquad ( 4 )$$

$${S}_{c , 2} = 1.5 {s}_{r} + {s}_{g} - 1.5 {s}_{b} \qquad ( 5 )$$

Then the rPPG signal is taken as the difference between the two

$$chrom = {S}_{c , 1} - \frac{σ ( {S}_{c , 1} )}{σ ( {S}_{c , 2} )} {S}_{c , 2} \qquad ( 7 )$$

with σ( . . . ) is the standard deviation of the signal. Note that the two projected signals were normalized by their maximum fluctuation. The CHROM method is derived to minimize the specular light reflection.

Next at 518 the projection matrix is applied to determine the pulse rate. For the projection matrix (PM) method the signal is projected to the pulsatile direction. Even though the three elements are not orthogonal, it was surprisingly found that this projection gives a very stable solution with better signal to noise than CHROM. To derive the PM method, the matrix elements of the intensity, specular, and pulsatile elements of the RGB signal are determined:

$${M}_{measured} = [ \begin{matrix}1 & 0.77 & 0.33 & 1 & 0.51 & 0.77 & 1 & 0.38 & 0.53\end{matrix} ] \qquad ( 8 )$$

The above matrix elements may be determined for example from a paper by de Haan and van Leest (G de Haan and A van Leest. Improved motion robustness of remote-ppg by using the blood volume pulse signature. Physiological Measurement, 35(9):1913, 2014). In this paper, the signals from arterial blood (and hence from the pulse) are determined from the RGB signals, and can be used to determine the blood volume spectra.

For this example the intensity is normalized to one. The projection to the pulsatile direction is found by inverting the above matrix and choosing the vector corresponding to the pulsatile. This gives:

$$pm = - 0.26 {s}_{r} + - 0.83 {s}_{g} - 0.50 {s}_{b} \qquad ( 9 )$$

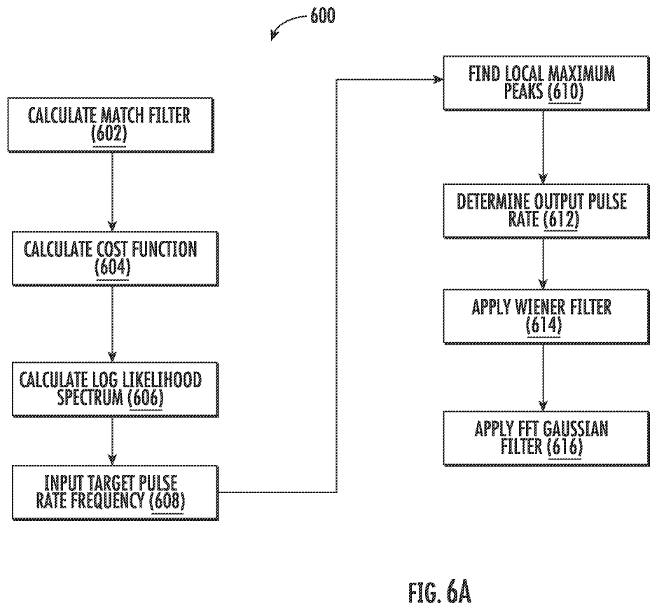

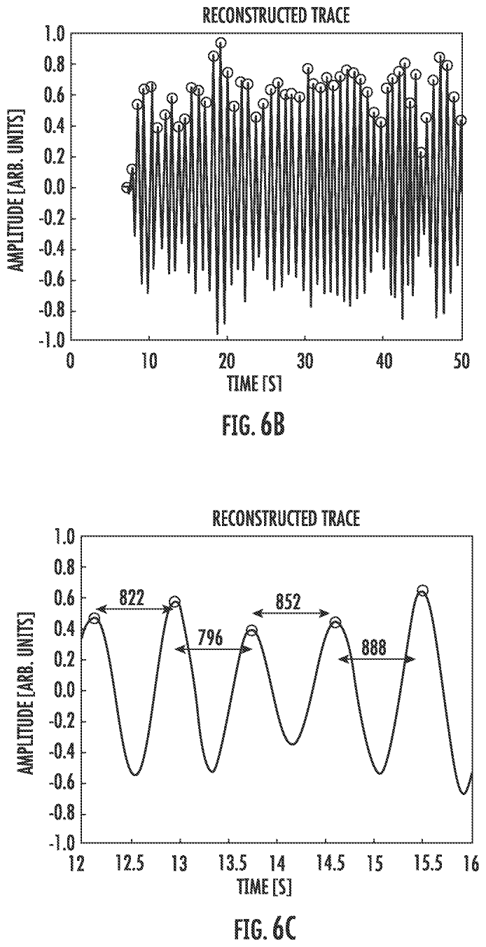

At 520, the two pulse rate results are cross-correlated to determine the rPPG. The determination of the rPPG is explained in greater detail with regard to FIG. 6.

FIG. 6A relates to a non-limiting exemplary method for pulse rate estimation and determination of the rPPG from optical data obtained of the face, while FIGS. 6B-6C relate to some results of this method. The method uses the output of the CHROM and PM rPPG methods, described above with regard to FIG. 5A, to find the pulse rate frequency vpr. This method involves searching for the mean pulse rate over the past Lalgo frames. The frequency is extracted from the output of a match filter (between the CHROM and PM), by using non-linear least square spectral decomposition with the application of a lock-in mechanism.

Turning now to FIG. 6A, in a method 600, the process begins at 602 by calculating the match filter between the CHROM and PM output. The match filter is simply done by calculating the correlation between CHROM and PM methods output. Next at 604, the cost function of a non-linear least squares (NLS) frequency estimation is calculated, based on a periodic function with its harmonics.

$$s ( n ) = {\sum }_{l = 0}^{L} [ {a}_{l} cos cos ( 2 π lvn ) - {b}_{l} sin sin ( 2 π lvn ) ] + ϵ ( n ) \qquad ( 10 )$$

In the above equation, x is the model output, al and bl are the weight of the frequency components, l is its harmonic order, L is number of orders in the model, v is the frequency, and (n) is the additive noise component. Then the log likelihood spectrum is calculated at 606 by adapting the algorithm given in Nielsen et. al (Jesper Kjor Nielsen, Tobias Lindstrøm Jensen, Jesper Rindom Jensen, Mads Grosbøll Christensen, and Søren Holdt Jensen. Fast fundamental frequency estimation: Making a statistically efficient estimator computationally efficient. Signal Processing, 135:188-197, 2017) in a computational complexity of O(N log N)+O(NL).

In Nielsen et. A, the frequency is set as the frequency of the maximum peak out of all harmonic orders. The method itself is a general method, which can be adapted in this case by altering the band frequency parameters. An inherent feature of the model is that higher order will have more local maximum peaks in the cost function spectra than lower order. This feature is used for the lock-in procedure.

At 608, the lock-in mechanism gets as input the target pulse rate frequency vtraget. Then at 610, the method finds all the local maximum peaks amplitude (Ap) and frequency (vp) of the cost function spectrum of order 1=L. For each local maximum, the following function is estimated:

$$f ( {A}_{p} , {v}_{p} , {v}_{traget} ) = \frac{{A}_{p}}{\vert {v}_{p} - {v}_{traget} \vert} \qquad ( 11 )$$

This function takes a balance between the signal strength and distance from the target frequency. At 610, the output pulse rate is set as local peak vp which maximize the above function f(Ap,vp,vtraget)

FIGS. 6B and 6C show an exemplary reconstructed rPPG trace (blue line), of an example run. The red circles show the peak R time. FIG. 6B shows the trace from run start at time t=0 s till time t=50 s. FIG. 6C shows a zoom of the trace, also showing RR interval times in milliseconds.

Next at 612-614, the instantaneous rPPG signal is filtered, with two dynamic filters around the mean pulse rate frequency (vpr): Wiener filter, and FFT Gaussian filter. At 612, the Wiener filter is applied. The desired target is sin(2πvprn), with n is the index number (representing the time). At 614, the FFT Gaussian filter aims to clean the signal around vpr, thus a Gaussian shape of the form

$$g ( ν ) = {e}^{{( \frac{ν - {ν}_{p r}}{{σ}_{g}} )}^{2}} \qquad ( 12 )$$

is used with σg as its width. As the name suggests, the filtering is done by transforming the signal to its frequency domain (FFT) and multiplying it by g (v) and transforming back to the time domain and taking the real part component.

The output of the above procedure is a filtered rPPG trace (pm) of length Lalgo with mean pulse rate of vpr. The output is obtained for each observed video frame and constructing the overlapping time series of pulse. These time series are then averaged to produce a mean final rPPG trace suitable for HRV processing. This is done using overlapping and addition of filtered rPPG signal (pm) using following formula (n represents time) from a paper by Wang et al (W. Wang, A. C. den Brinker, S. Stuijk, and G. de Haan. Algorithmic principles of remote ppg. IEEE Transactions on Biomedical Engineering, 64(7):1479-1491, July 2017):

$$t ( n - Lalgo + l ) = t ( n - Lalgo + l ) + w ( l ) pm ( l ) \qquad ( 13 )$$

with 1 is a running index between 0 and Lalgo; where w(i) is a weight function that sets the configuration and latency of the output trace. To obtain the necessary peaks (maxima that represents systolic peak), it is possible to construct RR intervals as distance in time. A series of RR intervals may be used to retrieve HRV parameters as statistical measurements in both time and frequency domains.

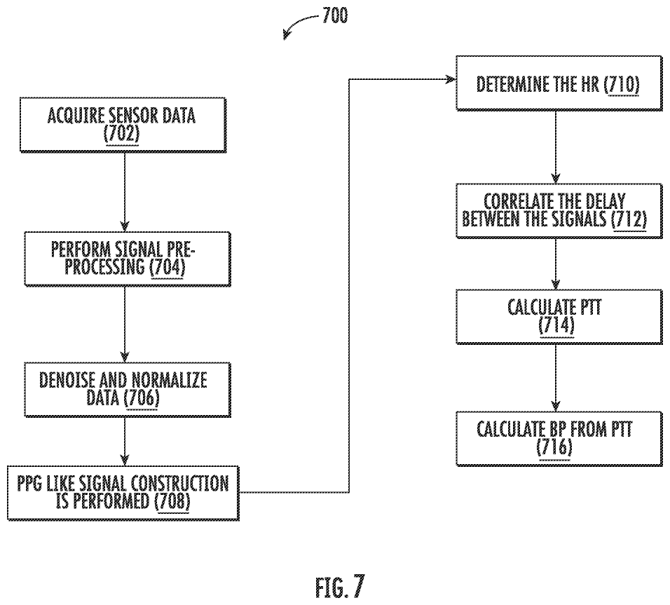

FIG. 7 relates to a non-limiting, exemplary implementation of a method for PTT determination and BP determination according to at least some embodiments of the present invention. The determined PTT is used for BP determination as shown.

As shown in a method 700, the method begins through sensor data acquisition at 702, preferably from a plurality of video cameras as previously described. However, it is not necessary to use the same type of sensors to provide signal. Optionally any suitable plurality of sensors may be used, whether the same or different, as long as suitable synchronization is provided. For example, preferably sampling rates are constant and known, and hardware provides time stamps. Then, the approach uses decimation/interpolation procedures to create a reliable signal that is sampled with the same sampling rate. It is preferred to use a synchronized clock for all sensors to prevent erroneous calculations.

Next, signal pre-processing is performed at 704 according to the previously described clock synchronization. Such signal processing includes aligning the sensor data in time to produce the same origin. This alignment is performed according to a combination of the hardware clock and the detected hardware delay between the sensors. In addition, the sensor data is interpolated/decimated to produce the same sampling rate. This can be done due to preliminary knowledge about the sampling rate of the sensors. Preferably a Hermite cubic or similar interpolation is used, rather than a linear or KNN interpolation. Such an interpolation is preferably selected to avoid spikes or other noise in the data. Optionally de-trending algorithms based on fast kernel density estimator (KDE) may be used.

Preferably the sensor data is de-noised to improve reliable values of SNR (signal to noise ratio) at 706. At this stage it may be necessary to return to the signal acquisition at 702. Preferably frequency based algorithms such as Wavelets and DCT are used with KDE to improve SNR. For a multi dimensional (multichannel like rPPG/PPG) sensor, PCA (principal component analysis) or ICA (independent component analysis) may be used to remove ambient and other noises.

After denoising, the sensor data is preferably normalized to produce the same level of magnitude. The sensor data is preferably also filtered between [0.5,4] Hz to produce bandwidth suitable for HR computation. Such filtering may be performed by using Butterworth N th-order bandpass. For simplicity N may be set to 3.

At 708, PPG like signal construction is performed. This stage differs for multidimensional sensors and single channel probes. For a single channel (one dimensional) sensor the procedure is as follows. First the coarse fundamental frequency is determined, for example by using Fundamental Frequency Estimation mechanism like NLS (Non-linear Least Square (NLS) frequency estimation) or PSD (power spectrum density) based methods. Next the proposed signal is preferably constructed as a sinusoidal wave. Next, using Wiener filtering procedure, re-construct the preserving initial phase and delay. Next the auto-correlation is computed.

The multidimensional case preferably includes a preliminary stage as follows. The projection matrix N×M matrix of data is computed, where N is the number of channels and M is the amount of data acquired for each channel into a 1×M vector. This vector may be produced as for the previously described CHROM, POS and PM used for rPPG. Since the procedure involves only means and variances related to each channel, the PCA and ICA also can be used. The PCA or ICA are preferred for channels with more than 3 dimensions or sensors, since the projection matrix becomes similar to PCA. The obtained signal is then processed as for the single channel.

At 710, the HR is determined. For example, optionally the fundamental frequency is calculated as HR from autocorrelation. The fine frequency is preferably determined using FFT based methodology around the proposed frequency for a given bandwidth (depending on sampling frequency). Optionally the determined HR is compared with another sensor. If they are sufficiently similar within a predefined threshold, the procedure preferably stops; otherwise new data is preferably acquired.

At 712, the delay between the different signals received from the plurality of sensors is calculated. For example, the delay may be calculated according to time delay estimation (TDE), which refers to finding the time differences of arrival between signals received at an array of sensors. A general signal model is:

$${r}_{i} [ n ] = {α}_{i} s [ n - {T}_{i} ] + {q}_{i} [ n ] , i - 1 , 2 … , M , n = 0 , 1 , … , N - 1$$

where is rᵢ[n] the received signal, s[n] is the signal-of-interest with α and Tᵢ being the gain/attenuation and propagation delay is the noise, at the i'th sensor.

There are M sensors, and at each sensor, N observations are collected. In a non-limiting example, M=2 and N may be large (normally 30˜60 sec observation).
Given above, the task of TDE is to estimate

$${T}_{i , j} = - {T}_{i , j} = {T}_{i} - {T}_{j} , i > j , i , j = 1 , 2 , … , M$$

After obtaining output for both sensors when the HR values agree, it is possible to compute the delay directly from autocorrelation functions or reconstructed PPG like signal obtained from the previous stage.
This process uses a combination of several methodologies, when applied to both autocorrelation and PPG signals. This combination produces a more robust result.
Gradient methods of time delay estimation are based on updating the delay by a vector that depends on information about the cost function to be minimized. The gradient algorithms involve the cost function as a second order Taylor's expansion around the proposed delay. Gradient algorithms based on the Gauss-Newton, steepest descent and least mean squares (LMS) method may be applied. Preferably the LMS method briefly described below is used.
The TDE considers two discrete-time signals incident on two sensors, which are sampled at time t=kTs, where Ts is the sampling period (assumed to be unity for simplicity) and expressed as previously described in following form

$$x ( k ) = {A}_{1 , s} ( k ) + v ( k ) \qquad ( 1 )$$

$$y ( k ) = {A}_{2 , s} ( k - T ) + n ( k ) \qquad ( 2 )$$

where s(k) corresponds to the noise-free source signal and s(k−T) is delayed, and A1 and A2 are their constant amplitudes. Where not explicitly stated, and without loss of generality, the value of A will be assumed to be unity in the sequel. The n(k) and v(k) are the uncorrelated zero-mean white Gaussian noise of variance σn2 and σv2, respectively.
The variable T represents the unknown time delay to be estimated, which is approximated to an integer closest to the true delay in the discrete-time model. The proposed method uses a cascade of a adapted filter W when the LMS is used to update its coefficients
The output z(k) of the W for x(k) input is given by

$$z ( k ) = {\sum }_{i = 0}^{M - 1} {w}_{i} ( k ) x ( k - i - {D}_{i} )$$

Where M is the length of W, D is a delay proposed to de-correlate the input signal, and wᵢ(k) are the filter coefficients at time k. The error term is given by

$$Z ( k ) = x ( k ) z ( k )$$

The desired sequence is given by

$$y ( k ) = {\sum }_{i = 0}^{M - 1} {h}_{i} ( k ) s ( k - i ) + n ( k )$$

Where h vector is the system impulse response. The output of the time delay estimator can be expressed as

$$\hat{y} ( k ) = {\sum }_{i = 0}^{M - 1} {g}_{i} ( k ) z ( k - i )$$

Where g is estimate of the system impulse response, and estimator error is given as

$$e ( k ) = y ( k ) - \hat{y} ( k )$$

The time delay estimate at K iteration is given as

$$\hat{D} ( k ) = arg max [ g ( k ) ]$$

The cross-correlation of two signals may also be used to estimate the time delay between the two signals, as the time at which the cross-correlation term is maximized corresponds to the time delay estimate. Briefly, for two sensors described below

$${x}_{1} ( t ) = {S}_{1} ( t ) + {n}_{1} ( t ) \qquad ( 1 )$$

$${x}_{2} ( t ) = α {S}_{1} ( t + D ) + {n}_{2} ( t ) \qquad ( 2 )$$

Using Generalized Cross Correlation, we can find the delay between each sensor. The techniques for this vary although the general application may be similar. A pre-filter specific to the method, is used in the frequency domain to ‘clean up’ the signal. It then undergoes cross correlation and a peak detection on that result to determine the point of maximum delay. The image below shows the continuous time model for the application of Generalized Cross Correlation (GCC). In this model, weighing functions, or pre-filters, are used to ‘clean’ the signal into a more useful form. For the purposes of this experiment, the methods used were the standard cross correlation, PHAT, and SCOT methods.

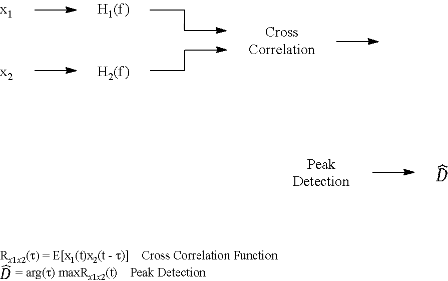

Unfortunately, correlation methods are typically not suitable with delay larger than a typical signal period if the sensor results are periodic, providing an incorrect delay, even with an opposite sign. Thus, additional information is needed, like the possible direction of the delay. For example, some requirements may be imposed, such that the delay from face to finger PPG's must be positive and the delay must be less than an appropriate mean RR interval (RR represents 1/HR) given in Hz.

Another way to compute time delay between two sensors may be found by determining the maximum of the probability density function of the delay. This procedure uses KDE terminology.

The proposed methodology provides an agreement between two different methodologies used to compute delay between sensors.

Next at 714, the PTT is calculated. As the blood flows through arteries, pressure waves propagate at a certain velocity called pulse wave velocity (PWV). The PWV depends on the elastic properties of both arteries and blood. The Moens-Korteweg equation defines PWV as a function of vessel and fluid characteristics [Bramwell J C, Hill A V. The Velocity of the Pulse Wave in Man. Proc. Royal Society for Experimental Biology & Medicine. 1922; 93:298-306; Ma, Y., Choi, J., Hourlier-Fargette, A., Xue, Y., Chung, H. U., Lee, J. Y., et al. (2018). Proc. Natl. Acad. Sci. U.S.A. 115:11144-11149. doi: 10.1073/pnas.1814392115].

$$P W V = \frac{L}{P T T} = \sqrt{\frac{E . h}{2 r ρ}} \qquad ( 3 )$$

where L is the vessel length, PTT is the time that a pressure pulse spends in transmitting through that length, p is the blood density, r is the inner radius of the vessel, h is the vessel wall thickness, and E is the elastic modulus of vascular wall.

From the PTT, the BP is calculated at 716. The Bramwell-Hills and Moens-Kortweg's equations give a logarithmic relationship between BP and the PTT. We can have the relationship of BP and the PTT represented as

$$B P = a * {log}_{e} ( P T T ) + b . \qquad ( 4 )$$

The equation above may be differentiated with respect to time [Sharma M., Barbosa K., Ho V., Griggs D., Ghirmai T., Krishnan S., Hsiai T., Chiao J. C., Cao H. Cuff-Less and Continuous Blood Pressure Monitoring: A Methodological Review. Technologies. 2017; 5:21. doi: 10.3390/technologies5020021] to obtain

$$B P = a * ( P T T ) + b * HR + c .$$

Here, a and b are subject-specific constants and they can be obtained through a regression analysis between the reference BP and the corresponding PTT [Chen W., Kobayashi T., Ichikawa S., Takeuchi Y., Togawa T. Continuous estimation of systolic blood pressure using the pulse arrival time and intermittent calibration. Med. Biol. Eng. Comput. 2000; 38:569-574. doi: 10.1007/BF02345755]. The mathematical relationship between BP and the Time Delay used in this work is a Linear Model that also exploits the HR as a second variable. Then, the model is:

$$BP = a * ( PTT ) + b * HR + c . \qquad ( 5 )$$

However, PTT in question (5) represents delay between ECG and PTT peaks and not relative PTT.

Changing regular PTT to a relative PTT preserves the question form and affects only the value of coefficients that can be easily calculated by regression analysis.

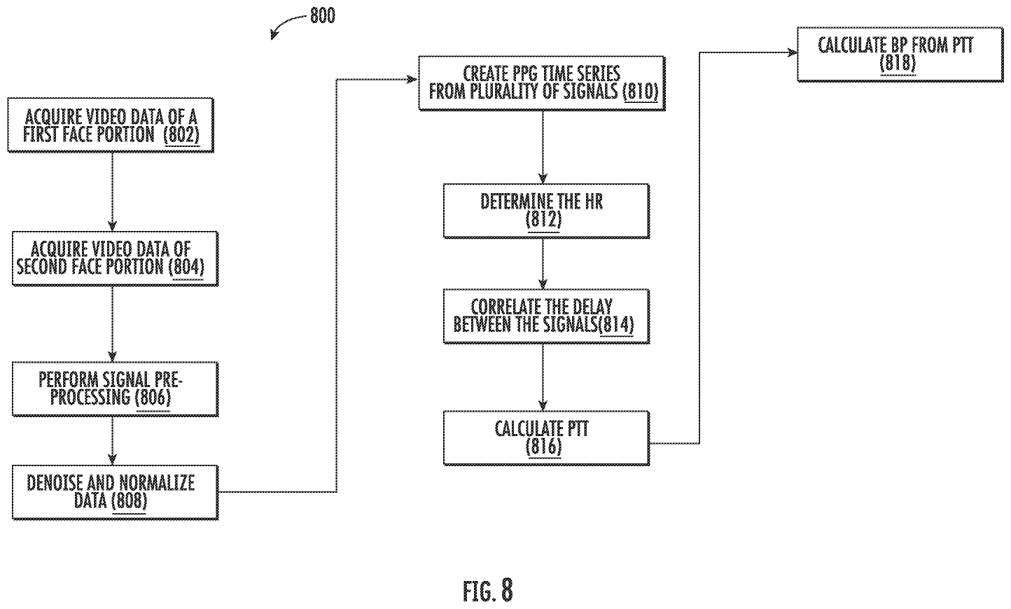

FIG. 8 relates to a non-limiting, exemplary implementation of a method for PTT determination and for BP determination with a plurality of face portions according to at least some embodiments of the present invention. The method of FIG. 8 is similar to that of FIG. 7, except that PPG signals are determined from video data from a plurality of face portions, such that the PPG signals from the plurality of portions are determined according to a delay, such that the PPG signals form a time series.

As shown, a method 800 begins at 802, by acquiring video data from a first face portion. The video data may be acquired as described herein, for example and without limitation according to the system of FIGS. 1A and 1B. The first face portion may comprise the entire face, or a portion thereof. Such a portion may comprise a region of the face, such as forehead, central face region or chin for example. Such a portion may also comprise one side of such a region, such that only part of that region is included, including without limitation a right portion of the forehead, a left portion of the forehead, a central portion of the forehead, a left cheek or a right cheek. A machine learning algorithm may be applied to locate the desired portion of the face, which may be the entirety of the face, according to facial boundary analysis as described herein.

Preferably sampling rates are constant and known, and hardware provides time stamps. It is possible to use decimation/interpolation procedures to create a reliable signal that is sampled with the same sampling rate. It is preferred to use a synchronized clock for all sensors to prevent erroneous calculations. In this non-limiting example, optionally the video data is obtained from one sensor, such as a front facing mobile camera, but at different times. In this case, the single sensor timing and sampling rate is predictable, but it is still necessary to synchronize the plurality of video data streams according to a clock.

At 804, video data of the second face portion is obtained, which as described above for the first face portion, may comprise the entire face, a region of the face, or a portion of such a region.

Next, signal pre-processing is performed at 806 according to the previously described clock synchronization. Such signal processing includes aligning the sensor data in time to produce the same origin. This alignment is performed according to a combination of the hardware clock and the detected hardware delay between the two video streams, as determined according to the time stamps. In addition, the video data may be interpolated/decimated to produce the same sampling rate, although alternatively the sampling rate may be inherently the same as being obtained from a single sensor (video camera). If needed, interpolation may be performed due to preliminary knowledge about the sampling rate of the sensors. Preferably a Hermite cubic or similar interpolation is used, rather than a linear or KNN interpolation. Such an interpolation is preferably selected to avoid spikes or other noise in the data. Optionally de-trending algorithms based on fast kernel density estimator (KDE) may be used.

Preferably the sensor data is de-noised to improve reliable values of SNR (signal to noise ratio) at 808. At this stage it may be necessary to return to the signal acquisition at 802 and/or 804. Preferably frequency based algorithms such as Wavelets and DCT are used with KDE to improve SNR. For a multi dimensional (multichannel like rPPG/PPG) sensor, PCA (principal component analysis) or ICA (independent component analysis) may be used to remove ambient and other noises.

After denoising, the sensor data is preferably normalized to produce the same level of magnitude. The sensor data is preferably also filtered between [0.5,4] Hz to produce bandwidth suitable for HR computation. Such filtering may be performed by using Butterworth N th-order bandpass. For simplicity N may be set to 3.

At 810, PPG like signal construction is performed from the plurality of signals. As such, the PPG like signal construction forms a time series, with signals being obtained according to a time offset or delay. This stage differs for multidimensional sensors and single channel probes. For a single channel (one dimensional) sensor the procedure is as follows. First the coarse fundamental frequency is determined, for example by using Fundamental Frequency Estimation mechanism like NLS (Non-linear Least Square (NLS) frequency estimation) or PSD (power spectrum density) based methods. Next the proposed signal is preferably constructed as a sinusoidal wave. Next, using Wiener filtering procedure, re-construct the preserving initial phase and delay. Next the auto-correlation is computed.

The multidimensional case preferably includes a preliminary stage as follows. The projection matrix N×M matrix of data is computed, where N is the number of channels and M is the amount of data acquired for each channel into a 1×M vector. This vector may be produced as for the previously described CHROM,POS and PM used for rPPG. Since the procedure involves only means and variances related to each channel, the PCA and ICA also can be used. The PCA or ICA are preferred for channels with more than 3 dimensions or sensors, since the projection matrix becomes similar to PCA. The obtained signal is then processed as for the single channel.

The pulse signals are preferably analyzed according to time, frequency and non-linear filters to support the determination of pulse waveform timing. In addition, a delay between time-series of the PPG signals of the plurality of face portions are preferably calculated in time interval, for example 30 seconds.

At 812, the HR (heart rate) is determined. For example, optionally the fundamental frequency is calculated as HR from autocorrelation. The fine frequency is preferably determined using FFT based methodology around the proposed frequency for a given bandwidth (depending on sampling frequency). Optionally the determined HR is compared with another sensor. If they are sufficiently similar within a predefined threshold, the procedure preferably stops; otherwise new data is preferably acquired.

At 814, the delay between the different signals received from the plurality of sensors, or from a single sensor with signals obtained at a plurality of different times, is calculated. For example, the delay may be calculated according to time delay estimation (TDE), which refers to finding the time differences of arrival between signals received at an array of sensors. A general signal model is:

$${r}_{i} [ n ] = {α}_{i} s [ n - {T}_{i} ] + {q}_{i} [ n ] , i - 1 , 2 … , M , n = 0 , 1 , … , N - 1$$

where is rᵢ[n] the received signal, s[n] is the signal-of-interest with α and Tᵢ being the gain/attenuation and propagation delay is the noise, at the i'th sensor.

There are M sensors, and at each sensor, N observations are collected. Alternatively, there is one sensor with N observations collected at each of a plurality of different times, for a plurality of different face portions. In a non-limiting example, M=2 and N may be large (normally 30˜60 sec observation).
Given above, the task of TDE is to estimate

$${T}_{i , j} = - {T}_{i , j} = {T}_{i} - {T}_{j} , i > j , i , j = 1 , 2 , … , M$$

After obtaining output for both sensors when the HR values agree, it is possible to compute the delay directly from autocorrelation functions or reconstructed PPG like signal obtained from the previous stage.
This process uses a combination of several methodologies, when applied to both autocorrelation and PPG signals. This combination produces a more robust result.
Gradient methods of time delay estimation are based on updating the delay by a vector that depends on information about the cost function to be minimized. The gradient algorithms involve the cost function as a second order Taylor's expansion around the proposed delay. Gradient algorithms based on the Gauss-Newton, steepest descent and least mean squares (LMS) method may be applied. Preferably the LMS method briefly described below is used.
The TDE considers two discrete-time signals incident on two sensors, which are sampled at time t=kTs, where Ts is the sampling period (assumed to be unity for simplicity) and expressed as previously described in following form

$$x ( k ) = {A}_{1 , s} ( k ) + v ( k ) \qquad ( 1 )$$

$$y ( k ) = {A}_{2 , s} ( k - T ) + n ( k ) \qquad ( 2 )$$

where s(k) corresponds to the noise-free source signal and s(k−T) is delayed, and A1 and A2 are their constant amplitudes. Where not explicitly stated, and without loss of generality, the value of A will be assumed to be unity in the sequel. The n(k) and v(k) are the uncorrelated zero-mean white Gaussian noise of variance σn2 and σv2, respectively.
The variable T represents the unknown time delay to be estimated, which is approximated to an integer closest to the true delay in the discrete-time model. The proposed method uses a cascade of a adapted filter W when the LMS is used to update its coefficients
The output z(k) of the W for x(k) input is given by

$$z ( k ) = {\sum }_{i = 0}^{M - 1} {w}_{i} ( k ) x ( k - i - {D}_{i} )$$

Where M is the length of W, D is a delay proposed to de-correlate the input signal, and wᵢ(k) are the filter coefficients at time k. The error term is given by

$$Z ( k ) = x ( k ) z ( k )$$

The desired sequence is given by

$$y ( k ) = {\sum }_{i = 0}^{M - 1} {h}_{i} ( k ) s ( k - i ) + n ( k )$$

Where h vector is the system impulse response. The output of the time delay estimator can be expressed as

$$\hat{y} ( k ) = {\sum }_{i = 0}^{M - 1} {g}_{i} ( k ) z ( k - i )$$

Where g is estimate of the system impulse response, and estimator error is given as

$$e ( k ) = y ( k ) - \hat{y} ( k )$$

The time delay estimate at K iteration is given as

$$\hat{D} ( k ) = arg max [ g ( k ) ]$$

The cross-correlation of two signals may also be used to estimate the time delay between the two signals, as the time at which the cross-correlation term is maximized corresponds to the time delay estimate. Briefly, for two sensors described below

$${x}_{1} ( t ) = {S}_{1} ( t ) + {n}_{1} ( t ) \qquad ( 1 )$$

$${x}_{2} ( t ) = α {S}_{1} ( t + D ) + {n}_{2} ( t ) \qquad ( 2 )$$

Using Generalized Cross Correlation, it is possible to find the delay between each sensor (or alternatively, between a plurality of different readings for one sensor). The techniques for this vary although the general application may be similar. A pre-filter specific to the method, is used in the frequency domain to ‘clean up’ the signal. It then undergoes cross correlation and a peak detection on that result to determine the point of maximum delay. The image below shows the continuous time model for the application of Generalized Cross Correlation (GCC). In this model, weighing functions, or pre-filters, are used to ‘clean’ the signal into a more useful form. For the purposes of this experiment, the methods used were the standard cross correlation, PHAT, and SCOT methods.

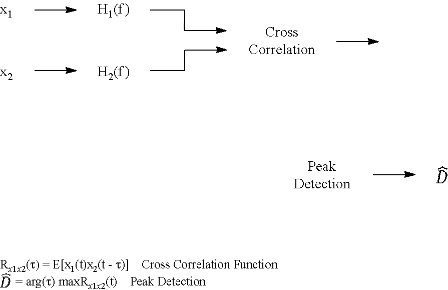

Unfortunately, correlation methods are typically not suitable with delay larger than a typical signal period if the sensor results are periodic, providing an incorrect delay, even with an opposite sign. Thus, additional information is needed, like the possible direction of the delay. For example, some requirements may be imposed, such that the delay from face to finger PPG's must be positive and the delay must be less than an appropriate mean RR interval (RR represents 1/HR) given in Hz.

Another way to compute time delay between two sensors may be found by determining the maximum of the probability density function of the delay. This procedure uses KDE terminology.

Hence, these time-series of the PPG, along with the delay, can be used to derive PTT values through the natural delay between the pulse signals detected at the two different regions of the face.

Next at 816, the PTT is calculated. As the blood flows through arteries, pressure waves propagate at a certain velocity called pulse wave velocity (PWV). The PWV depends on the elastic properties of both arteries and blood. The Moens-Korteweg equation defines PWV as a function of vessel and fluid characteristics [Bramwell J C, Hill A V. The Velocity of the Pulse Wave in Man. Proc. Royal Society for Experimental Biology & Medicine. 1922; 93:298-306; Ma, Y., Choi, J., Hourlier-Fargette, A., Xue, Y., Chung, H. U., Lee, J. Y., et al. (2018). Proc. Natl. Acad. Sci. U.S.A. 115:11144-11149. doi: 10.1073/pnas.1814392115].

$$PWV = \frac{L}{PTT} = \sqrt{\frac{E \cdot h}{2 r ρ}} \qquad ( 3 )$$

where L is the vessel length, PTT is the time that a pressure pulse spends in transmitting through that length, p is the blood density, r is the inner radius of the vessel, h is the vessel wall thickness, and E is the elastic modulus of vascular wall.

From the PTT, the BP is calculated at 716. The Bramwell-Hills and Moens-Kortweg's equations give a logarithmic relationship between BP and the PTT. We can have the relationship of BP and the PTT represented as

$$BP = a * {log}_{e} ( PTT ) + b . \qquad ( 4 )$$

The equation above may be differentiated with respect to time [Sharma M., Barbosa K., Ho V., Griggs D., Ghirmai T., Krishnan S., Hsiai T., Chiao J. C., Cao H. Cuff-Less and Continuous Blood Pressure Monitoring: A Methodological Review. Technologies. 2017; 5:21. doi: 10.3390/technologies5020021] to obtain

$$BP = a * ( PTT ) + b .$$

Here, a and b are subject-specific constants and they can be obtained through a regression analysis between the reference BP and the corresponding PTT [Chen W., Kobayashi T., Ichikawa S., Takeuchi Y., Togawa T. Continuous estimation of systolic blood pressure using the pulse arrival time and intermittent calibration. Med. Biol. Eng. Comput. 2000; 38:569-574. doi: 10.1007/BF02345755]. The mathematical relationship between BP and the Time Delay used in this work is a Linear Model that also exploits the HR as a second variable. Then, the model is:

$$BP = a * ( PTT ) + b * HR + c . \qquad ( 5 )$$

However, PTT in question (5) represents delay between ECG and PTT peaks and not relative PTT.

Changing regular PTT to a relative PTT preserves the question form and affects only the value of coefficients that can be easily calculated by regression analysis.

After obtaining the above measurement of PTT, the PTT measurement may be used for more accurate determination of blood pressure (BP), including without limitation better determination of BP variability, at 818, for example as described herein.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A method for calculating a blood pressure (BP) for a subject, the method comprising obtaining optical data from multiple regions of a face of the subject with a camera; analyzing the optical data to select data related to the face with a computational device in communication with said camera; detecting optical data from a skin of the face, determining a time series from the optical data by collecting the optical data until an elapsed period of time has been reached and then calculating the time series from the collected optical data for the elapsed period of time; calculating the PTT (pulse transit time) from the time series; and calculating the BP using the calculated PTT; wherein the optical data comprises video data, and wherein said obtaining said optical data from a specific region of the skin of the face comprises obtaining video data of the face of the subject; wherein said obtaining said video data of said fingertip comprises obtaining video data of the skin of said fingertip of the subject by placing said fingertip on said camera.

2. (canceled)

3. (canceled)

4. The method of claim 1, wherein said camera comprises a plurality of mobile phone cameras, wherein said obtaining said optical data further comprises obtaining video data from said plurality of mobile phone cameras, wherein optical data from the face is obtained with a first mobile phone camera and optical data from the finger is obtained with a second mobile phone camera.

5. The method of claim 4, wherein the subject places the finger on a rear facing mobile phone camera and the face of the subject is located in front of a front facing mobile phone camera for obtaining video data of said finger and of said face.

6. The method of claim 5, wherein said fingertip on said mobile phone camera further comprises activating a flash associated with said mobile phone camera to provide light.

7. The method of claim 6, wherein each of said video data from said first and second mobile phones are analyzed to provide pulse signal information from said finger and face skin.

8. The method of claim 7, wherein a delay between pulse signals determined from said first and second mobile phones is determined according to synchronization through a hardware clock; wherein said pulse signals are synchronized and are then interpolated to produce the same sampling rate.

9. (canceled)

10. The method of claim 1, wherein said computational device is physically separate from, but in communication with, said mobile phone camera.

11. The method of claim 1, wherein said computational device comprises a mobile communication device.

12. The method of claim 1, wherein said detecting said optical data from said skin of the face comprises determining a plurality of face boundaries, selecting the face boundary with the highest probability and applying a histogram analysis to video data from the face; and wherein said detecting said optical data from said skin of the finger comprises determining a plurality of skin boundaries, selecting the skin boundary with the highest probability and applying a histogram analysis to video data from the skin.

13. The method of claim 12, wherein said determining said plurality of face boundaries comprises applying a multi-parameter convolutional neural net (CNN) to said video data to determine said face boundaries; and wherein said determining said plurality of skin boundaries comprises applying multi-parameter convolutional neural net (CNN) to said video data to determine said skin boundaries.

14. (canceled)

15. (canceled)

16. (canceled)

17. (canceled)

18. The method of claim 1, wherein said determining the PTT further comprises combining meta data with measurements from said optical data from said skin of the face and from said skin of the finger, wherein said meta data comprises one or more of weight, age, height, biological gender, body fat percentage and body muscle percentage of the subject.

19. The method of claim 1, further comprising determining the PTT from at least one additional physiological signal; determining at least one additional physiological signal at least from the PTT; or a combination thereof; wherein said physiological signal is selected from the group consisting of stress, blood pressure, breath volume, and pSO2 (oxygen saturation).

20. (canceled)

21. (canceled)

22. The method of claim 1, further comprising before calculating the PTT, denoising and normalizing said pulse signals; filtering said pulse signals; performing PPG like signal construction, determining a heart rate (HR) from said PPG like signal construction and calculating the PTT from said HR; and calculating blood pressure from the PTT.

23. (canceled)

24. (canceled)

25. (canceled)

26. (canceled)

27. (canceled)

28. (canceled)

29. (canceled)

30. (canceled)

31. A system for calculating a PTT (pulse transit time) for a subject, the system comprising: a camera for obtaining optical data from a face and from a fingertip of the subject, a user computational device for receiving optical data from said camera, wherein said user computational device comprises a processor and a memory for storing a plurality of instructions, wherein said processor executes said instructions for analyzing the optical data to select data related to the face and the fingertip of the subject, detecting optical data from a skin of the face and a skin of the fingertip, determining a time series from the optical data by collecting the optical data until an elapsed period of time has been reached and then calculating the time series from the collected optical data for the elapsed period of time; and calculating the PTT from the time series; wherein said memory is configured or storing a defined native instruction set of codes and said processor is configured to perform a defined set of basic operations in response to receiving a corresponding basic instruction selected from the defined native instruction set of codes stored in said memory; whereon said memory stores a first set of machine codes selected from the native instruction set for analyzing the optical data to select date related to the face of the subject, a second set of machine codes selected from the native instruction set for detecting optical data from a skin of the face, a third set of machine codes selected from the native instruction set for determining a time series from the optical data by collecting the optical data until an elapsed period of time has been reached and then calculating the time series from the collected optical data for the elapsed period of time; and a fourth set of machine codes selected from the native instruction set for calculating the physiological signal from the time series; wherein said memory further comprise a fifth set of machine codes selected from the native instruction set for detecting said optical data form said skin of the face comprises determining a plurality of face boundaries, a sixth set of machine codes selected from the native instruction set for selecting the face boundary with the highest probability and a seventh set of machine codes selected from the native instruction set for applying a histogram analysis to video data from the face; wherein said memory is configured for storing a defined native instruction set of codes and said processor is configured to perform a defined set of basic operations in response to receiving a corresponding basic instruction selected from the defined native instruction set of codes stored in said memory; wherein said memory stores a stores a ninth set of machine codes selected from the native instruction set for analyzing the optical data to select data related to the fingertip of the subject, a tenth set of machine codes selected from the native instruction set for detecting optical data from a skin of the fingertip, a eleventh set of machine codes selected from the native instruction set for determining a time series from the optical data by collecting the optical data until an elapsed period of time has been reached and then calculating the time series from the collected optical data for the elapsed period of time; and a twelfth set of machine cods selected from a native instruction set for detecting said optical data from said skin of the fingertip comprises determining a plurality of fingertip boundaries, a fourteenth set of machine codes selected from the native instruction set for selecting the fingertip boundary with the highest probability and a fifteenth set of machine codes selected from the native instruction set for applying a histogram analysis to video data from the fingertip.

32. (canceled)

33. (canceled)

34. The system of claim 31, wherein said memory further comprises an additional set of machine codes selected from the native instruction set for applying a multi-parameter convolutional neural net (CNN) to said video data to determine said face boundaries, for applying a multi-parameter convolutional neural net (CNN) to said video data to deterring said fingertip boundaries, or a combination thereof.

35. (canceled)

36. (canceled)

37. (canceled)

38. The system of claim 31, wherein the fingertip is pressed against the camera for obtaining optical data so only skin detection is performed, rather than fingertip detection; and wherein said camera comprises a mobile phone camera and wherein said optical data is obtained as video data from said phone camera.

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. A system for calculating a PTT (pulse transit time) from a subject, the system comprising: a rear facing camera for obtaining optical data from a finger of the subject, a user computational device for receiving optical data from said camera, wherein said user computational device comprises a processor and a memory for storing a plurality of instructions, wherein said processor executes said instructions for analyzing the optical data to select data related to the face of the subject, detecting optical data from a skin of the finger, determining a time series from the optical data by collecting the optical data until an elapsed period of time has been reached and then calculating the time series from the collected optical data for the elapsed period of time; and calculating the PTT from the time series.

51. (canceled)

52. (canceled)

53. A system for calculating a blood pressure (BP) for a subject, the system comprising: a camera for obtaining optical data from specific regions of a subject's face; a computational device in communication with the camera, the computational device including a memory for storing instructions and a processor for executing said instructions, wherein said instructions comprise instructions for analyzing variations in skin color and texture associated with blood flow by:
analyzing the optical data to select data related to the face to determine a time series from the optical data by collecting the optical data until an elapsed period of time has been reached and then calculating the time series from the collected optical data for the elapsed period of time, wherein said time series reflects optical changes from the skin of the face, correlating with heartbeats, and wherein said analyzing the optical data comprises analyzing the optical data under variable lighting conditions for the camera when collecting the optical data;
calculating a PTT (pulse transit time) from the time series; and
calculating the BP using the calculated PTT.
